(19) United States
(12) Patent Application Publication
(10) Pub. No.: US 2002/0118339 A1
(43) Pub. Date: Aug. 29, 2002

(54) Eye tracking display apparatus

(75) Inventor: Anthony Cyril Lowe, Braishfeild (GB)

Correspondence Address: IBM Corp, IP Law, Route 134/Kitchawan Road, Yorktown Heights, NY, 10598, US

(73) Assignee: International Business Machines Corporation, Armonk, NY

(21) Appl. No.: 10/076,763

(22) Filed: Feb. 14, 2002

(30) Foreign Application Priority Data
Feb. 23, 2001 (GB) GB 0104451.0

(51) Int. Cl. A61B 3/14

(52) U.S. Cl. 351/209

(57) ABSTRACT

Display apparatus comprising a display screen for the display of an image to be viewed by a user. The display apparatus has one or more eye trackers for monitoring the eye motion of the user. The motion detected is processed to calculate an effective displacement of the image caused by the eye motion. The image displayed on the screen is moved to compensate for the effective displacement caused by the eye motion. The display apparatus may have an image displayed on the screen which has a background which is light in color, the area of the display screen surrounding the displayed image may be dark in color. The periphery of the displayed image is graduated so as to be the same as the area of the display screen surrounding the displayed image at the outer periphery and the same as the background of the image at the inner periphery.

### FIELD OF THE INVENTION

The present invention relates to display apparatus and in particular to display apparatus where the displayed image tracks movements of the eyes of the user.

### BACKGROUND OF THE INVENTION

Nystagmus is an eye condition characterized by spontaneous, oscillatory, short and jerky movements of the eyes. Nystagmus causes the point at which an image is focussed on the retina to jitter, while still staying within the central portion of the retina. Nystagmus is believed to be the brain's natural mechanism to prevent saturation and exhaustion of individual receptor cells in the retina. Subsequent post-retinal neural processing completely removes the motions induced by nystagmus from the perceived scene. However, in some people, this post-retinal neural processing either does not occur or is not effective. Sufferers from this condition whose post-retinal neural processing either does not work or is not effective are often completely debilitated by it. However, in some cases, activities that usually require normal vision, such as using a computer, are still possible, though difficult and tiring.

The problems which these sufferers have has been traced to the presence of a cerebellar-vestibular dysfunction which prevents ocular fixation and sequential scanning of letters and words in a proper manner. Specifically, during sequential scanning or normal reading, letters and words are disordered, and letter and word scrambling or blurring results.

For example, the biggest or first letter of the word is often fixated first during the slow right-to-left phase of the nystagmus movement. The rapid left-to-right phase often skips over several letters or a whole word until another letter is automatically fixated and scrambling or blurring results. The person, therefore, confuses letters and words which differ only or mainly in spatial placement, i.e. b=d=p=q, a=e, c=u, m=w, saw=was, no=on, and the like. This confusion of letters and words results in reading difficulties. Thus, it would be desirable to have a display apparatus in which the nystagmus movements of the eye were compensated for.

### SUMMARY OF THE INVENTION

Accordingly, the present invention provides a display apparatus comprising: a display screen for the display of an image to be viewed by a user; one or more eye trackers for monitoring the eye motion of the user; and a processor for calculating the effective displacement of the image caused by the eye motion of the user; wherein the image displayed on the screen is moved so as to compensate for the effective displacement caused by the eye motion of the user.

In an advantageous embodiment, the display apparatus comprises one or more eye trackers, and a processor for monitoring the distance of the user from the display screen and wherein the processor compensates for changes in the distance of the user from the display screen.

In a first embodiment, the one or more eye trackers monitor eye separation.

In a second embodiment, the one or more eye trackers monitor the differences in the angle subtended by the eyes at one or more detectors.

Alternative embodiments may further comprise a filter responsive only to oscillatory, short and/or jerky movements of the eyes of the user.

Advantageously, the display apparatus further comprises a graphical user interface for adjustment of the gain of the processing system in converting eye motion of the user into movement of the image on the display screen.

In a particularly advantageously embodiment, the image displayed on the screen has a background which is, say light in color, whereas the area of the display screen surrounding the displayed image is, say dark in color, and the periphery of the displayed image is graduated so as to be the same as the area of the display screen surrounding the displayed image at the outer periphery, and to be the same as the background of the image at the inner periphery.

### BRIEF DESCRIPTION OF THE DRAWINGS

Embodiments of the invention will now be described, by way of example, with reference to the accompanying drawings, in which:

FIG. 1 is a schematic diagram of the measurement of image displacement caused by eye motion;

FIG. 2 is a block diagram showing an example of elements of the present invention; and

FIG. 3 is a particularly advantageous embodiment of the invention using a graduated periphery area.

### DETAILED DESCRIPTION OF THE INVENTION

The present invention provides display apparatus in which eye motion is detected and employed to move the image presented on the display in synchrony with the eye motion, producing an acceptably stationary image on the retina.

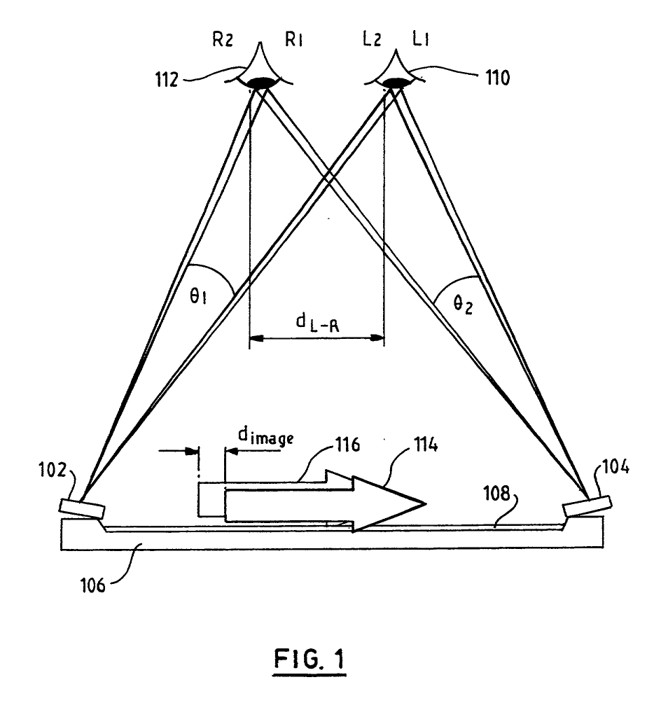

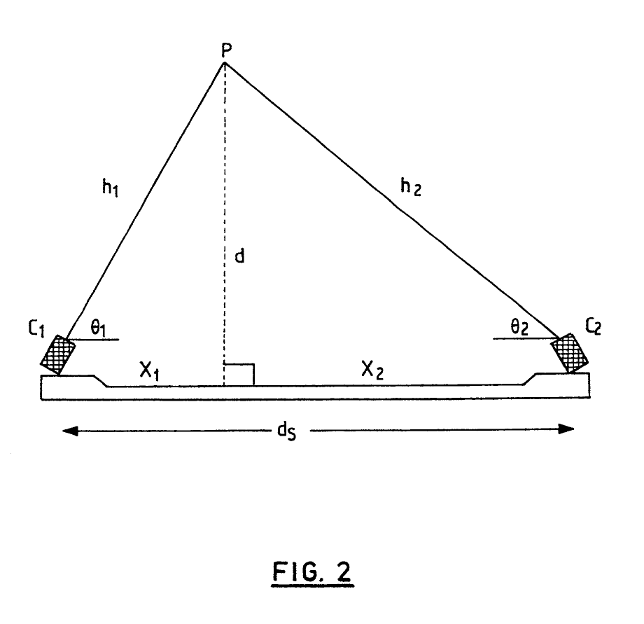

Referring to FIG. 1, eye trackers 102, 104 are shown located on display apparatus 106. Apparatus according to the present invention may comprise a single eye tracker 102 or it may comprise two or more eye trackers 102, 104. The display apparatus has a screen 108 on which images are displayed for viewing by a user. Also shown in FIG. 1 are the left 110 and right 112 eyes of a user viewing the image in the display screen.

The one or more eye trackers 102, 104 are used to convert measured eye motion into an equivalent displacement of the image on the display screen 108. Two trackers 102, 104 are necessary if it is desired to correct for changes in viewing distance during use. The separation of the eyes 110, 112, d_{L-R} is monitored so that changes in viewing distance can be monitored. The position (direction of view) of the eyes 110, 112 is also monitored. As an alternative to monitoring the separation of the eyes 110, 112, the difference, θ1, θ2 in the angles subtended by the eyes 110, 112 at one or the other eye trackers 102, 104 (depending on the mode of operation of the trackers) may be monitored in order to changes in viewing distance. In some embodiments, both techniques are combined together in a single display system.

In the example of FIG. 1, the initial position of the left 110 and right 112 eyes is denoted by (L1,R1). The position of the left 110 and right 112 eyes after eye motion is denoted by (L2,R2). From the angular motion of the eyes 110, 112 and the eye-display separation, the effective displacement of the display image 114, d_{image}, and its direction φ, or alternatively, the x,y coordinates of the original and displaced images, can be calculated.

Referring to FIG. 2, a block diagram of an embodiment of the present invention is shown. An image is displayed on display screen 108 by a processor 202. The movement of the user's eyes is tracked by means of eye trackers 102, 104. In the example of FIG. 1, the image is initially positioned on the display screen 108 at a position 114. When the eye trackers 102, 104 detect movement of the user's eyes 110, 112, processor 202 calculates a new position 116 for the image on the display screen 108 to bring the image as viewed by the user back to the original position on the retina now that eye movement has occurred. The calculated position may be identified in terms of a distance d_{image} in a direction φ or it may be identified in terms of x and y coordinates. position of the image displayed on the display screen 108 is then moved from its original position 114 by the distance d_{image} in the direction φ to a new position 116 on the display screen 108 to bring it back to the original position on the retina now that eye movement has occurred. Thus the involuntary eye movement by the viewer of the screen will largely be compensated for. If the x,y coordinates of the original and displaced images were calculated, then the image is displaced so as to compensate for this displacement.

Because of differences between eye displacement and image displacement for a single observer caused, for example, by changes in iris dilation changes and because of differences between observers, a Graphical User Interface function 204 can optionally be provided that enables the viewer to adjust the system gain to produce an acceptably stationary image.

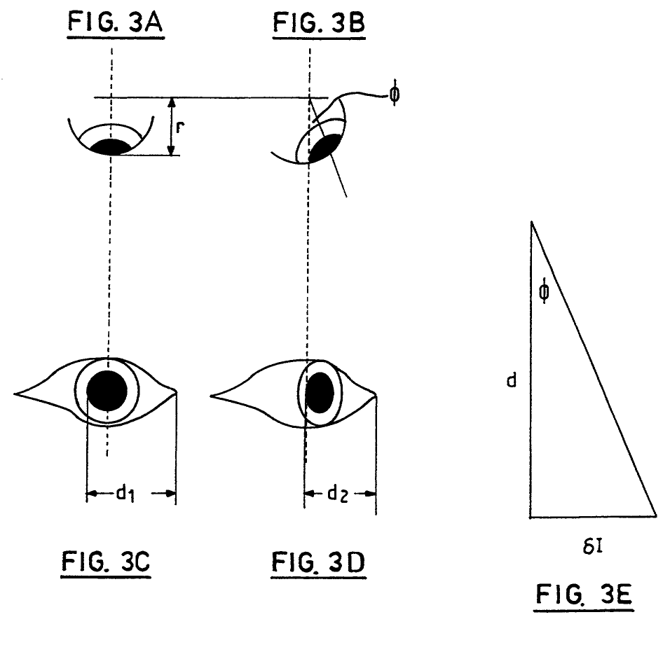

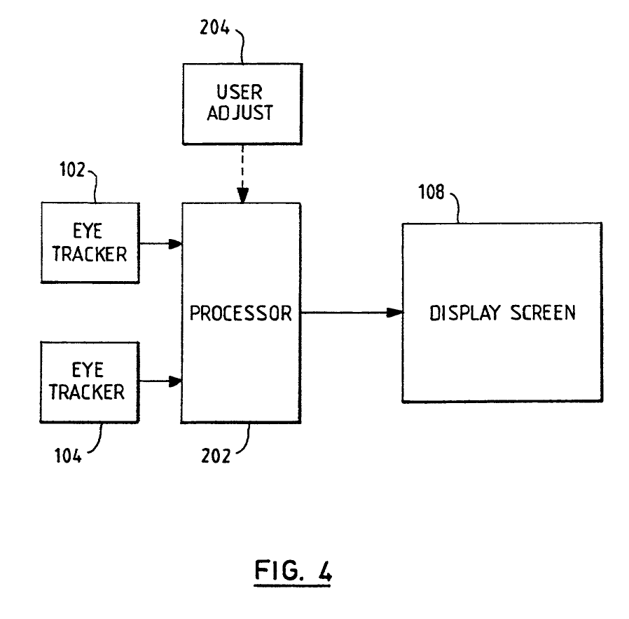

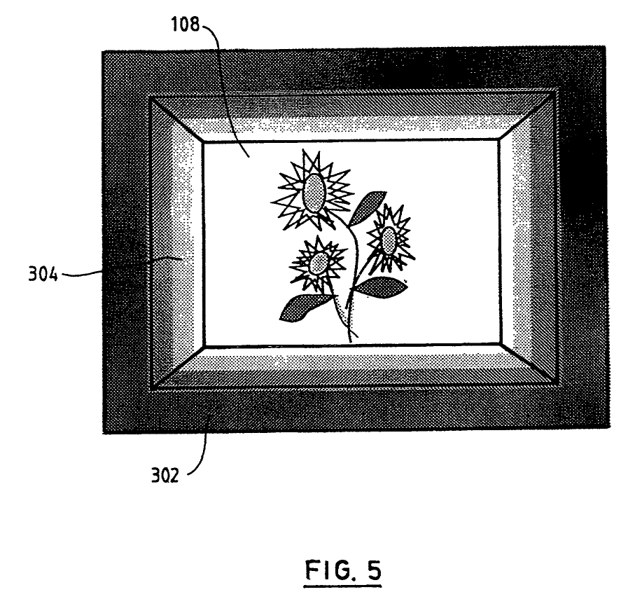

Referring to FIG. 3, the display bezel 302 and its surroundings remains acceptably stationary when the image 108 on the display is moved and this may produce undesirable consequences for the viewer such as fatigue or effects similar to nystagmus itself and the like. Such effects can be alleviated by choosing a dark bezel color such as stealth black and displaying an acceptably stationary border 304 around the periphery of the display that will match the bezel 302 color at its outer extremity and gradually fade into the displayed image at its inner boundary. Thus the between the image that is being moved to create an acceptably stationary image on the retina and the stationary image plus the bezel will be diffuse.

Although illustrative embodiments and its advantages have been described in detail hereinabove, they have been described by way of example and not by way of limitation. Various changes, substitutions and alterations can be made in the illustrative embodiments without departing from the concepts, breadth, scope and spirit of the present invention.

What is claimed is:

1. A display apparatus comprising:
a display screen for the display of an image viewed by a user;
at least one eye tracker for monitoring eye motion of the user; and
a processor for calculating an effective displacement of the image caused by the eye motion of the user;
wherein the image displayed on the screen is moved so as to compensate for the effective displacement caused by the eye motion of the user.

2. A display apparatus as claimed in claim 1, further comprising a processor for monitoring a distance of the user from the display screen and wherein the processor compensates for changes in the distance of the user from the display screen.

3. A display apparatus as claimed in claim 1, wherein said at least one eye tracker monitor eye separation.

4. A display apparatus as claimed in claim 1, further comprising at least one detector, and said at least one eye tracker monitor the differences in the angle subtended by the eyes at said at least one detector.

5. A display apparatus as claimed in claim 1, further comprising a filter responsive to at least one movement of a group of movements including oscillatory, short and jerky movements of the eyes of the user.

6. A display apparatus as claimed in claim 1, further comprising a graphical user interface for adjustment of the gain of the processor in converting eye motion of the user into movement of the image on the display screen.

7. A display apparatus as claimed in claim 1, wherein:
the image displayed on the screen has a background which is light in color;
the area of the display screen surrounding the displayed image is dark in color; and
the periphery of the displayed image is graduated so as to be the same as an area of the display screen surrounding the displayed image at an outer periphery and the same as a background of the image at an inner periphery.

8. A display apparatus as claimed in claim 1, wherein the periphery of the displayed image is graduated so as to be a same color as a first color of an area of the display screen surrounding the displayed image at an outer periphery, and to be the same color as a second color of an area of a background of the image at an inner periphery.
